(19) United States
(12) Patent Application Publication
(10) Pub. No.: US 2026/0053823 A1
(43) Pub. Date: Feb. 26, 2026

(54) PREVENTATIVE MOUTHWASH CONTAINING A SUB-ANTIMICROBIAL ORAL DOSAGE OF DOXYCYCLINE, METHOD OF PREVENTING COVID INFECTION USING THE MOUTHWASH, AND METHOD OF FORMULATING THE MOUTHWASH

(71) Applicant: Bruce Roseman, White Plains, NY (US)

(72) Inventor: Bruce Roseman, White Plains, NY (US)

(21) Appl. No.: 19/014,137

(22) Filed: Jan. 8, 2025

Related U.S. Application Data
(63) Continuation-in-part of application No. 18/814,513, filed on Aug. 24, 2024, now abandoned.

(51) Int. Cl. A61K 31/65; A61K 8/33; A61K 8/42; A61Q 11/00

(52) U.S. Cl. CPC A61K 31/65; A61K 8/33; A61K 8/42; A61Q 11/00; A61K 2800/92

(57) ABSTRACT

A preventative mouthwash includes doxycycline as an active ingredient, and sufficient solvent (such as distilled or purified water) to dissolve a sub-antimicrobial oral dosage (such as between 1 and 40 milligrams) of the doxycycline into a gargling dosage (such as between 5 and 20 milliliters) of the mouthwash. The mouthwash may further include a flavoring agent (such as benzaldehyde) to improve taste. A method of preventing COVID infection includes gargling the preventative mouthwash, and repeating the gargling twice a day (such as every 12 hours) for several (such as between 5 and 10, like 7) consecutive days. A method of formulating the preventative mouthwash includes dissolving doxycycline hyclate in purified water to create a liquid solution having doxycycline as an active ingredient, and dissolving a flavoring agent in the liquid solution to create the mouthwash. Here, a gargling dosage of the mouthwash contains a sub-antimicrobial oral dosage of the doxycycline.

### CROSS-REFERENCE TO RELATED APPLICATION

The present application is a continuation-in-part and claims the benefit of U.S. application Ser. No. 18/814,513, filed on Aug. 24, 2024, the entire content of which is incorporated herein by reference.

### FIELD

Embodiments of the disclosure relate to a doxycycline-based mouthwash for use in preventing coronavirus disease 2019 (COVID-19 or COVID) infection. The preventative mouthwash contains a sub-antimicrobial oral dosage of the doxycycline. Further embodiments relate to a method of preventing COVID infection using the mouthwash. Still further embodiments relate to a method of formulating the preventative mouthwash.

### BACKGROUND

COVID and its variants have produced worldwide epidemics. Traditional vaccine development entails significant investment in time, resources, and effort, offering only temporary protection against evolving viral strains like COVID. Doxycycline is a proven broad-spectrum antibiotic when delivered at a sufficiently high (cytotoxic) dose to induce its antibiotic properties. However, multiple such doses over time can lead to bacterial resistance issues such as bacterial overgrowth.

### SUMMARY

According to an embodiment of the disclosure, a preventative mouthwash is provided. The mouthwash includes doxycycline as an active ingredient, and sufficient solvent to dissolve a sub-antimicrobial oral dosage of the doxycycline into a gargling dosage of the mouthwash.

In an aspect, the solvent includes distilled or purified water.

In an aspect, the sub-antimicrobial oral dosage is between 1 milligram (mg) and 40 mg of the doxycycline, and the gargling dosage is between 5 milliliters (ml) and 20 ml of the mouthwash.

In a further aspect, the sub-antimicrobial oral dosage is between 1 mg and 4 mg of the doxycycline, and the gargling dosage is about 5 ml of the mouthwash.

In an aspect, the mouthwash further includes a flavoring agent to improve taste.

In a further aspect, the flavoring agent includes benzaldehyde.

In an aspect, the mouthwash further includes a preservative to improve shelf life.

In an aspect, the mouthwash further includes a pH adjuster to maintain proper pH.

According to another embodiment of the disclosure, a method of preventing COVID infection is provided. The method includes gargling a preventative mouthwash, and repeating the gargling twice a day for several consecutive days. The mouthwash includes doxycycline as an active ingredient, and sufficient solvent to dissolve a sub-antimicrobial oral dosage of the doxycycline into a gargling dosage of the mouthwash.

In an aspect of the method, several consecutive days is between 5 and 10 consecutive days.

In a further aspect of the method, several consecutive days is 7 consecutive days.

In an aspect of the method, twice a day is every 12 hours.

In an aspect of the method, the sub-antimicrobial oral dosage is between 1 milligram (mg) and 40 mg of the doxycycline, the gargling dosage is between 5 milliliters (ml) and 20 ml of the mouthwash, and gargling the preventative mouthwash includes gargling the gargling dosage for at least 30 seconds followed by spitting out the gargled mouthwash.

In a further aspect of the method, the sub-antimicrobial oral dosage is between 1 mg and 4 mg of the doxycycline, and the gargling dosage is about 5 ml of the mouthwash.

According to yet another embodiment of the disclosure, a method of formulating a preventative mouthwash is provided. The formulation method includes dissolving doxycycline hyclate in distilled or purified water to create a liquid solution including doxycycline as an active ingredient, and dissolving a flavoring agent in the liquid solution to create the preventative mouthwash. A gargling dosage of the mouthwash includes a sub-antimicrobial oral dosage of the doxycycline.

In an aspect of the formulation method, the flavoring agent includes benzaldehyde.

In an aspect of the formulation method, the method further includes dissolving a preservative in the liquid solution to improve shelf life.

In an aspect of the formulation method, the method further includes dissolving a pH adjuster in the liquid solution to maintain proper pH.

In an aspect of the formulation method, the sub-antimicrobial oral dosage is between 1 milligram (mg) and 40 mg of the doxycycline, and the gargling dosage is between 5 milliliters (ml) and 20 ml of the mouthwash.

In a further aspect of the formulation method, the sub-antimicrobial oral dosage is between 1 mg and 4 mg of the doxycycline, and the gargling dosage is about 5 ml of the mouthwash.

The above and other aspects and embodiments of the present disclosure will become apparent from the following description read in conjunction with the accompanying drawings, in which like or similar reference numerals designate like or similar elements.

### BRIEF DESCRIPTION OF DRAWINGS

To better understand the present disclosure, reference is made to the following drawings in which:

FIG. 1 is a table of a set of seven high-risk patients used to study the efficacy of a comparable mouthwash at preventing COVID infection;

FIG. 2 is a flow diagram of an example method of preventing COVID infection using a mouthwash containing a sub-antimicrobial oral dosage of doxycycline, according to an embodiment; and

FIG. 3 is a flow diagram of an example method of formulating a preventative mouthwash containing a sub-antimicrobial oral dosage of doxycycline, according to an embodiment.

### DETAILED DESCRIPTION

Embodiments of the disclosure are directed to a doxycycline-based mouthwash for preventing COVID-19 infection that uses preemptive measures targeting viral invasion at the cellular level. Embodiments of the proposed mouthwash inhibit penetration of the virus into host cells and inhibit cleaving of replicated polypeptides of the virus at particular genetic markers essential for viral replication in the host cells. This impedes virus penetration and reproduction, potentially averting widespread infection and long-term COVID complications. Embodiments of the proposed mouthwash include administering the mouthwash in sub-cytotoxic levels of doxycycline to avoid unintended antimicrobial effects.

As discussed above, traditional vaccine development entails significant investment in time, resources, and effort, offering only temporary protection against evolving viral strains like COVID. The recurrent emergence of viral variants underscores the limitations of conventional vaccination strategies in addressing evolving threats. [4] Viral infection prevention strategies often rely solely or primarily on reactive vaccine development. Likewise, while antibody drugs can also help prevent viral infection, they must be administered intravenously and are limited in their ability to handle new variants of COVID. [4] Further, attempts to use mouthwash to prevent COVID infection have been either ineffective or only demonstrated short-term effectiveness. [8]

In response to these and other such shortcomings, embodiments of the present disclosure provide for a doxycycline-based preventative mouthwash that proactively targets the fundamental mechanisms of viral invasion and replication. This helps prevent COVID infection and avoid potentially harmful and expensive treatment options later. Embodiments of the proposed mouthwash use sub-antimicrobial dosages of doxycycline to inhibit viral fusion and entry into the host cells, such as by inhibiting human host proteases essential for COVID fusion and entry into the host cells. Some embodiments further inhibit cleaving of replicated polypeptides at key genetic markers crucial for viral replication in the host cells. Embodiments of the proposed mouthwash inhibit the fundamental mechanisms of COVID invasion and replication, and without the unintended side effects of cytotoxic levels of doxycycline. As such, embodiments of the proposed mouthwash disrupt the initial stages of infection, potentially curbing the spread of viral strains and mitigating long-term COVID sequelae. Embodiments can be particularly effective therapeutics for high-risk groups (elderly or comorbidities, such as obesity) at initial stages of infection. [15]

Embodiments of the present disclosure take advantage of the statistical unlikelihood of the COVID virus being able to mutate in such a way as to alter its fundamental method of entry or invasion into the host cells, or to alter its fundamental method of replication and assembly within the host cells. Both stages rely on proteases whose functions are inhibited by doxycycline. This is a far more efficient treatment option versus spending countless resources (e.g., time, money) attempting to figure out what the next strains of COVID will eventually look like and responding with more specifically targeted treatment options at that time. Provided that the invasion and replication of follow-on viruses follow the same basic viral pathogeny of other COVID strains, embodiments of the present disclosure will be just as effective on future strains. Accordingly, the use of the term “COVID” throughout is intended to cover past, present, and future virulent strains of viruses that descended from SARS-COV-2.

Doxycycline is a proven broad-spectrum antibiotic (with anti-inflammatory and cardioprotective properties) that also blocks protein biosynthesis and inhibits penetration of viruses into host cells, both of which hamper the replication of the virus in the host body. [1] Doxycycline is also an effective antiviral antibiotic against RNA viruses such as COVID-19. [1] Doxycycline treatment has also been shown to reduce the need for ICU admission after onset of COVID-19 infection, and without serious side effects. [2] Doxycycline is a safe, inexpensive, and widely available oral treatment for addressing COVID-19 infection. [2] Doxycycline exhibits potent inhibition of metalloproteinases, which are associated with the severity of COVID-19. [2] Doxycycline is an example of an oral small-molecule drug that can be repurposed to fight COVID-19 infections. Such drugs offer a more efficient treatment for new COVID variants versus traditional vaccine or antibody drug regimes. [3]

Embodiments of a doxycycline-based mouthwash provide for a general treatment of COVID invasion and replication, and whose mechanism remains essentially independent of genetic mutations to the virus. This effectively prevents COVID infection throughout the body. Embodiments of the mouthwash prevent cellular invasion and replication of the COVID virus. Using doxycycline as an active ingredient in prophylactic dosage levels, embodiments of the disclosure prevent, inactivate, and/or inhibit the protease enzymes inside the host cell (and necessary for COVID penetration and assembly) from becoming active. As long as the protease enzymes are prevented from becoming activated there will be no invasion and no replication of the new mutant virus.

In the discussion that follows, milliliters or ml represents a liquid measurement equivalent to cubic centimeters or cc. Further, while some of the discussion below refers to 5 ml of a mouthwash as an oral dosage, the gargling dosage of doxycycline mouthwash can vary between embodiments (such as between 5 ml and 20 ml), with corresponding varying concentrations of doxycycline in the mouthwash to effect equivalent oral dosages of doxycycline, as would be apparent to one of ordinary skill. For instance, 800 milligrams (mg) of doxycycline per liter (L) of mouthwash represents a 0.08 % concentration of doxycycline (by weight). If such a mouthwash had a corresponding gargling dosage of 5 ml, then an oral dosage of 4 mg of doxycycline would be present in the gargling dosage of the mouthwash. To make an equivalent oral dosage of doxycycline for a mouthwash having a 20 ml gargling dosage, the doxycycline concentration would have to be cut by 4 (to 0.02 %) to deliver the same 4 mg oral dosage of doxycycline in the gargling dosage of the mouthwash.

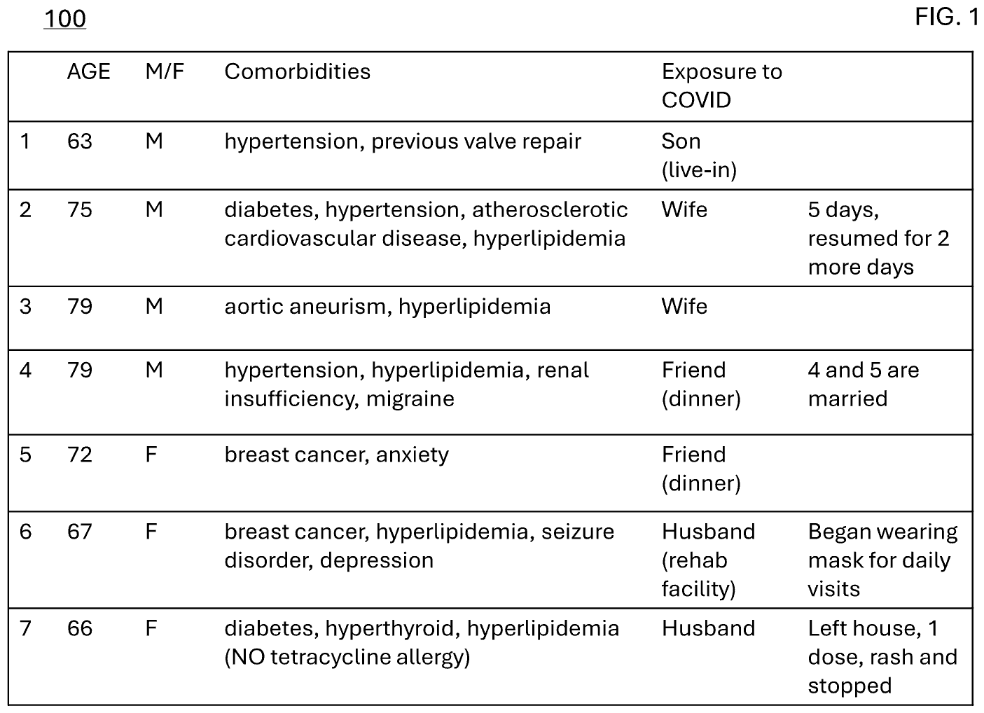

FIG. 1 is a table 100 of a set of seven high-risk patients used to study the efficacy of a comparable mouthwash at preventing COVID infection. With reference to FIG. 1, the seven high-risk patients (elderly with comorbidities) had frequent contact with relatives or friends exhibiting COVID systems (and positive test results for COVID). Each of these patients was administered 5 ml of an antimicrobial doxycycline-based mouthwash twice a day for seven to ten days. Specifically, the mouthwash included 400 mg of doxycycline per 5 ml of mouthwash, or 8 % concentration by weight. In all cases, the patients did not come down with COVID symptoms; they did not get sick. This helps demonstrate the efficacy of the mouthwash in preventing COVID infection. It should be noted that the focus of the study was use of a doxycycline mouthwash for preventing the spread of COVID (in particular, not getting sick) among high-risk patients with known exposure to COVID. The focus was not for treating actual COVID infections.

Invasion and duplication of the COVID virus are nearly completely independent of genetic changes in the virus. The host cell's protease enzymes are used to break down the long polypeptides of attachment proteins and replicated genomes of any new virulent COVID virus. These are crucial portions of the penetration and assembly stages of the new virus. By inhibiting this breakdown, invasion and replication of the new virus are effectively inhibited, which prevents infection.

For example, if the spike protein of a new COVID virus is not broken down into smaller peptide units after attachment to the host cell ACE2 (or CD147) receptor, fusion with the host cell membrane and subsequent entry of the COVID virion into the host cell will be arrested. Further, if the replicated genome (long polypeptide) of the entered virion is not properly broken down into smaller peptides needed to assemble new virions, the virus will not be able to replicate itself from inside the host cell. As such, when the doxycycline inactivates the protease enzymes of targeted host cells, there will be no opportunity for the virus to enter the cells and no opportunity to replicate even if they did enter.

Among other uses, doxycycline functions as a protease inhibitor. See, e.g., [2], [11], and [12], for how this property works in arresting COVID, cancer, and dengue fever progressions. Without sufficient proteases, virus infection shuts down. For instance, while the COVID virion uses the spike protein to attach to the host cell at the ACE2 (or possibly CD147) receptor during the first stage of a virus (attachment), the second stage (penetration) requires proteases (e.g., furin) to cleave the spike protein into functional units that carry out the fusion of the virion with the host cell membrane. Doxycycline inhibits these proteases, which inhibits penetration (invasion) of the attached virion into the host cell (penetration inhibition).

In a similar fashion, proteases are also needed to cleave the virion's RNA polypeptide after the third stage (uncoating) and fourth stage (replication of the uncoated viral genome into the polypeptide in the host cell) of the virus. This cleaving turns the replicated polypeptide into useful proteins needed to assemble more virions inside the host cell. Doxycycline inhibits these proteases as well, which inhibits the fifth stage (assembly of useful virions) and effectively prevents replication of the virions in the host cell (assembly inhibition). Without assembly, the sixth stage (release) of the virus becomes moot.

As such, sufficient doxycycline prevents COVID viral infection by inhibiting (through protease inhibition) the penetration and assembly stages of the virus. In addition, these inhibitions are (for practical purposes) independent of any mutations in the virion RNA, which also prevents infection of COVID sequelae. Further, by keeping these doxycycline dosages below antibacterial (cytotoxic) levels, potentially harmful and unintended side effects of doxycycline are reduced or eliminated.

In some embodiments, the mouthwash formulation does not cause any bacterial overgrowth. Administering any antibacterial agent (such as doxycycline) at any dose and for any length of time can lead to bacterial overgrowth. This is because of the emergence and growth of bacterial strains that are resistant to the antibacterial agent. However, in some embodiments of the proposed mouthwash, this will not happen as the amount of doxycycline used in the mouthwash combined with the frequency and period of administering the mouthwash is too small (such as sub-cytotoxic) to lead to overgrowth.

In further detail, the minimal inhibitory concentration (MIC) of doxycycline, which is the lowest concentration of the drug that inhibits the growth of bacteria, varies depending on the bacterial species. The MIC of doxycycline ranges from 0.1 to 4.0 μg per ml (micrograms per milliliter), which is equivalent to 0.1 to 4.0 mg per L (milligrams per liter). To achieve therapeutic antibacterial effectiveness for doxycycline, blood concentrations should be maintained above the MIC of the targeted pathogen(s). In example embodiments, however, therapeutic blood levels of doxycycline are kept below these ranges in order to reduce or minimize any unintended antibacterial effects.

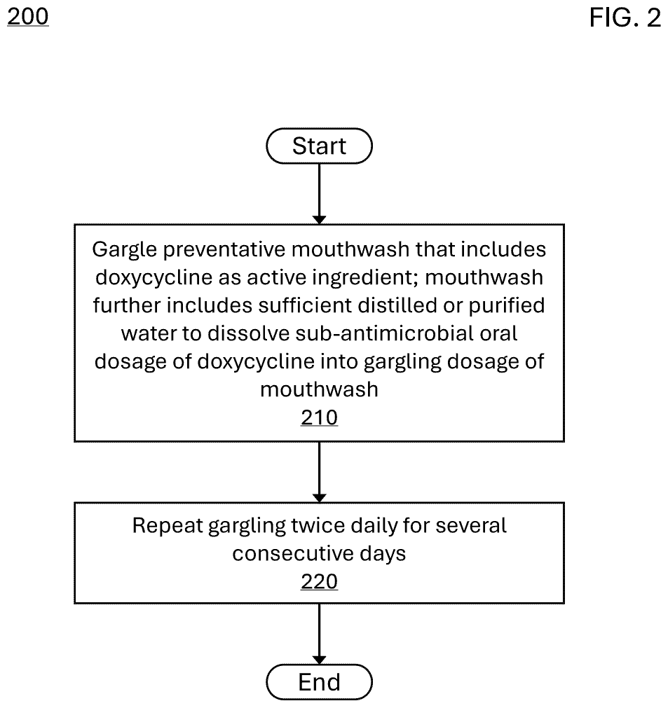

FIG. 2 is a flow diagram of an example method 200 of preventing COVID infection using a mouthwash containing a sub-antimicrobial oral dosage of doxycycline, according to an embodiment. The method 200 includes gargling 210 (e.g., for at least 30 seconds, such as between 30 and 60 seconds) a preventative mouthwash including doxycycline as an active ingredient, followed by spitting out the gargled mouthwash. The mouthwash further includes sufficient distilled or purified water to dilute a gargling dosage (e.g., 5 ml) of the mouthwash to a sub-antimicrobial oral dosage of doxycycline (e.g., between 0.02% and 0.08% of doxycycline by weight, such as 0.04%, for the 5 ml gargling dosage). The method 200 further includes repeating 220 the gargling twice a day (such as every 12 hours) for several consecutive days (such as between 5 and 10 consecutive days, as in 7 consecutive days).

In an example embodiment, to estimate the blood sum concentration of doxycycline, 400 mg (milligrams) of doxycycline hyclate is dissolved in 1000 ml of solution (e.g., distilled or purified water) to produce a concentration of 0.4 mg/ml, or 0.04 % by weight. 5 ml of this solution (i.e., 2 mg of doxycycline) is then gargled every 12 hours for seven consecutive days. In other embodiments, the solution is gargled twice a day for several consecutive days, such as between five and 10 consecutive days. In still other embodiments, the gargling dosage is varied (such as between 5 ml and 20 ml of the mouthwash), with corresponding adjustments in the doxycycline concentration to maintain an equivalent oral dosage (e.g., 2 mg) of doxycycline from the gargling dosage of the mouthwash.

Actual intake of doxycycline depends on several factors, including absorption rate from the oral cavity, availability, and systemic uptake. Since the gargle is done twice a day, the daily dose in the above examples is 4 mg. The availability of doxycycline swallow is typically around 80%. However, for gargling and not swallowing, absorption from the oral mucosa would likely be significantly lower, potentially around 10 to 20% based on the limited absorption surface and relatively short contact time.

Accordingly, assuming a 15% rate of absorption from gargling 5 minutes, then 4 mg of doxycycline per day times 0.15 absorption equals 0.6 mg per day of absorbed doxycycline. The half-life of doxycycline is approximately 18 to 22 hours for steady concentration. As such, considering a half-life of 20 hours and using the simplified steady state formula (serum concentration equals fraction absorbed times the dose over the clearance of the drug), clearance with doxycycline is roughly 0.1 L (liter) per hour per kilogram (kg) for a 70 kg adult. Using these approximations, steady concentration equals 0.6 mg per day over 7 L times 24 hours a day, or roughly 0.0036 mg/liter. That is, the estimated blood serum concentration of doxycycline when gargling 5 ml with this 4 mg/ml doxycycline solution, every 12 hours for seven days, would be approximately 0.0036 mg/L. This is a very low concentration (and well below cytotoxic levels of doxycycline), reflecting the minimal systemic absorption from gargling without swallowing.

Since the lowest MIC for doxycycline is 0.1 μg/ml (or mg/L), this 2 mg oral dosage of doxycycline can be increased by a factor of 20 (0.0036 mg/L×20 =0.072 mg/L) and still stay below the 0.1 mg/L lowest MIC level of doxycycline. As such, twice-daily gargling of mouthwash having between 1 mg and 40 mg of doxycycline per gargling dosage (i.e., 0.02% to 0.8% doxycycline by weight for a mouthwash having 5 ml as a gargling dosage) will result in a sub-antimicrobial oral dosage of the doxycycline being delivered. Equivalent doxycycline concentrations for other gargling dosages of the mouthwash can be used to provide the same sub-antimicrobial oral dosages of the doxycycline.

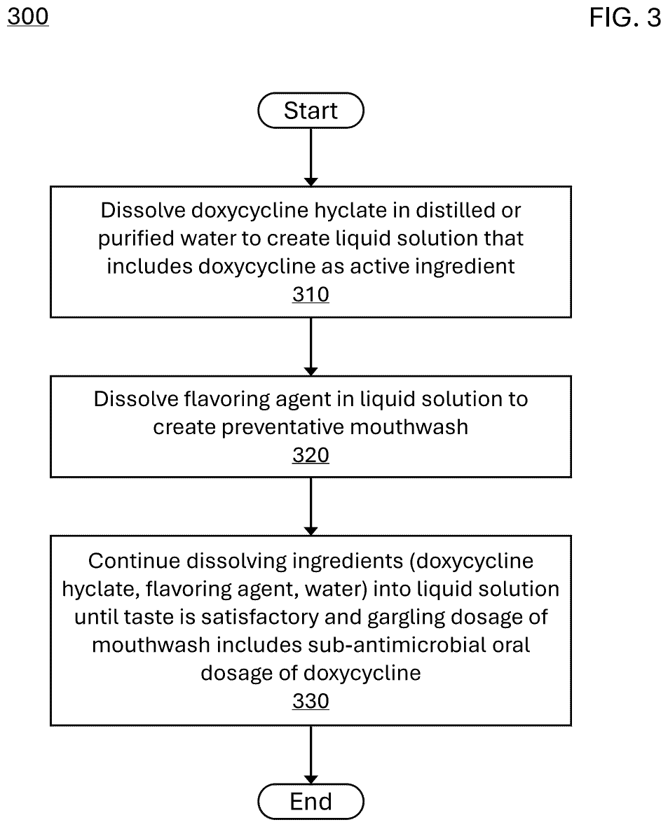

FIG. 3 is a flow diagram of an example method 300 of formulating a preventative mouthwash containing a sub-antimicrobial oral dosage of doxycycline, according to an embodiment. The mouthwash is a doxycycline liquid solution. The mouthwash is directed at preventing COVID infection to effectively prevent the spread of the next COVID virus. The method 300 includes dissolving 310 doxycycline hyclate in distilled or purified water to create a liquid solution including doxycycline as an active ingredient. The method 300 further includes dissolving 320 a flavoring agent (e.g., benzaldehyde) in the liquid solution to create the preventative mouthwash. In addition, the method 300 includes continuing 330 to dissolve ingredients (e.g., water, doxycycline hyclate, flavoring agent) into the liquid solution until the taste is satisfactory and the gargling dosage of the mouthwash (e.g., 5 ml) contains a sub-antimicrobial oral dosage of doxycycline (e.g., between 1 mg and 4 mg of doxycycline, which is equivalent to between 0.02% and 0.08% concentration of doxycycline by weight for the 5 ml gargling dosage).

Given the rate of nucleotide mutations, it is only a matter of time before the COVID virus mutates into a more virulent strain. However, because these mutations do little to affect the overall COVID life cycle, the new strain will likely remain susceptible to the same inhibitory properties of doxycycline as previous COVID strains before the mutations.

In further detail, there is no known treatment to eradicate the COVID virus, which has been around for longer than recorded history. As such, we will likely never be able to eradicate the virus's genus. The virus's life cycles (including those of its recent descendants, MERS-CoV, SARS-CoV-1, and SARS-CoV-2) are dependent on the presence of certain protease enzymes. While we do not know the genomic makeup of the next pathogenic strain of COVID, its life cycle will almost certainly be dependent on the same protease enzymes. Vaccines and monoclonal antibodies are very strain specific. Accordingly, it is not practical to develop and administer specific vaccines or monoclonal antibodies as a complete worldwide virus treatment plan.

However, embodiments of doxycycline mouthwash help prevent the spread of the different COVID strains by acting more like a generic deterrent. That is, they inhibit some of the basic proteases necessary for the COVID life cycles. Embodiments of the present disclosure operate nearly independently of any specific COVID genome by inhibiting proteases needed by the virus to penetrate through the host cell membranes after attachment to the host cell receptor sites. Embodiments of doxycycline mouthwash further inhibit proteases needed by the virus inside the host cell that reconfigure the proteins necessary for replication of the virus inside the host cell. By preventing the virus from entering the host cells at the sites of entry (receptor sites), embodiments of the present disclosure work immediately to prevent viral invasion by inhibiting those enzymes needed by the virus to penetrate the host cell and reproduce.

Embodiments of doxycycline work as a generic defense against all COVID strains since they inhibit the proteases common to the life cycles of any genetic mutation of COVID (i.e., the proteases needed for viral penetration and duplication of any strain of COVID). As such, they work as a defense on the common denominator of the COVID strains (genomic sequences). In more detail, the virions of a COVID strain first attach themselves to the ACE2 receptors of the host cell membranes. Such host cells are concentrated in the oropharynx. From here, proteases of the host cells assist with penetration of the virions through the cell membranes. Further proteases of the host cells assist with replication of the virions after the virions' RNA cores are uncoated. Embodiments of doxycycline mouthwash provide an immediate start on antiviral therapy by suppressing infection (especially host cell penetration) at the most likely entry point of the body. This effectively prevents the exponential growth of viral load needed to establish infection by pinpointing the site of viral invasion and inhibiting the proteases needed for early penetration and replication in the body at that site.

Embodiments of the present disclosure deliver all the benefits of antiviral antiproteases without the drawbacks of other such pharmaceuticals. For instance, Paxlovid™ is underutilized not so much for its efficacy as its side effects. These include its unpleasant taste and its large potential for drug-to-drug interactions (and hence preclusion) with commonly used drugs. By contrast, some embodiments of the present mouthwash use a simple flavoring agent (such as benzaldehyde) to improve taste and doxycycline (particularly in sub-antimicrobial dosages) has a relatively small number of drug-to-drug interactions and that are well understood.

Embodiments of doxycycline mouthwash work immediately because they start to work when the mouthwash enters the oral cavity. This prevents viral entry into the cells most likely to receive the virus. Example embodiments are flavored (such as cherry flavored) to provide for comfortable administration. The potential dosage of doxycycline administered is low enough to provide for practically no drug-to-drug interactions. The safety profile of doxycycline has already been established. Photosensitivity reactions are also unlikely at the intended dosages. The mouthwash works by systemic absorption to effect overall changes in the microbiome.

In an example formulation of the mouthwash, doxycycline hyclate is used as it is more soluble in water compared to doxycycline base. Water (for example, distilled or purified water) is used to supply the liquid portion of the liquid solution. In addition, other additives are used as needed (e.g., flavoring agents to improve taste, preservative to improve shelf life, and pH adjusters to maintain proper pH). Formulation equipment can include measuring devices (such as beakers and pipettes), mixing devices (such as a magnetic stirrer), containers for storage (ideally amber colored to protect from light), and personal protective equipment (such as gloves and masks).

For an example sub-antimicrobial doxycycline concentration of 0.04%, 400 mg of doxycycline are needed for every liter of solution (equivalently 0.4 mg per ml). We first need to determine the equivalent amount of doxycycline hyclate. Doxycycline and doxycycline hyclate have slightly different molecular weights, so the correct amount of doxycycline hyclate needs to be determined. Doxycycline hyclate is about 102% of the weight of doxycycline due to the addition of the hydrochloride salt. Therefore, to convert 400 mg of doxycycline to an equivalent amount of doxycycline hyclate, you need 400 mg times 1.02 equals 408 mg of doxycycline hyclate. We next need to dissolve the 408 mg of doxycycline hyclate into a suitable solvent to maintain the desired concentration (e.g., 1 liter of liquid solution to maintain a 0.04% doxycycline concentration). The choice of solvent depends on the use of the solution and any solubility considerations, such as flavoring (e.g., cherry) and other additives. Sufficient solvent is added (along with the other additives) to produce 1 liter of liquid solution with the 0.04% doxycycline concentration.

In more detail, the doxycycline hyclate should be thoroughly mixed in the solvent until it is completely dissolved. The other additives can then be added and mixed, with enough solvent added afterwards to bring the total volume of the liquid solution to one liter. As a verification, the actual concentration of doxycycline can be determined in the liquid solution to be sure the final product meets the requirements. The storage of the final product should be suitable for doxycycline, usually in a cool dark place to prevent degradation. It should be noted that the 0.04% doxycycline concentration is just an example. In other embodiments, different sub-cytotoxic levels of doxycycline are used, such as between 0.02% and 0.08% doxycycline concentration by weight. Still other embodiments can use up to 0.8% doxycycline concentration (again, for the 5 ml gargling dosages, with corresponding adjustments to the doxycycline concentration for other gargling dosages).

In further detail, doxycycline is a tetracycline derivative with broad-spectrum antimicrobial activities that is used as an effector substance in inducible gene-expression systems. For example, in an investigation of the antiviral activity of doxycycline against vesicular stomatitis virus (VSV) infection in cultured H1299 cells, doxycycline at concentrations of 1.0-2.0 μg/ml significantly inhibited VSV replication and the VSV-induced cytopathic effect in dose-dependent manners. [5] This suggests that doxycycline may have broader activity in inhibiting viral replication, in addition to its well-defined bacteriostatic activity. Doxycycline exerted its antiviral effect at the early-mid stage of VSV infection, suggesting that it did not interfere with VSV infectivity, adsorption, or entry into target cells. These results indicate that doxycycline can inhibit VSV infection and may therefore have potential applications for the treatment of viral infections.

In [15], doxycycline was shown to promote expression of zinc-finger antiviral protein (ZAP), which inhibits the replication of many viruses. Moreover, doxycycline is a safer early treatment alternative to azithromycin for high-risk groups, such as elderly and comorbidities. [15]

Moreover, in [6] and [7], repurposing of safer, established medications that may have antiviral activity is proposed as a possible approach for treatment of earlier-stage COVID-19. In particular, tetracycline and its derivatives (such as doxycycline) are proposed as nontraditional antibiotics with a well-established safety profile, potential efficacy against viral pathogens such as dengue fever and chikungunya, and that may regulate important infection and replication pathways of COVID-19. More specifically, four cases of COVID patients with pulmonary disease and that are treated with doxycycline exhibited rapid clinical improvement with no safety issues. The authors note that doxycycline is an attractive repurposed drug treatment candidate for COVID-19 infection. In addition, in [15], it was noted that doxycycline is an effective early treatment for COVID infection in high-risk groups (such as elderly or nursing home residents).

In further detail, doxycycline is well known to inhibit metalloproteinases (MMPs), in particular MMP-9, which is likely required for initial viral entry into the cell. [6] Further, doxycycline inhibits interleukin (IL)-6, which together with MMPs are key regulators of cytokine storms associated with severe viral pneumonitis. [6] Doxycycline is also an established ionophore, helping transport zinc intracellularly, with increased cellular concentrations of zinc shown in vitro to inhibit coronavirus replication. [6] In addition, doxycycline inhibits nuclear factor (NF)-κB, which may lower risk of viral entry due to direct inhibition of the DPP4 cell surface receptor. [6] Doxycycline in sub-antimicrobial dosages inhibits expression of CD147/EMMPRIN, which may be necessary for entry into T lymphocytes. [6] Further, doxycycline has the potential to inhibit papain-like proteinase (PLpro) and 3C-like main protease (3CLpro or Mpro), viral proteins essential to the viral replication cycle. [6]

In some embodiments, doxycycline is administered as a mouthwash in sub-antimicrobial dosages to help prevent COVID infection by inhibiting viral replication and cell invasion, and without reaching cytotoxic levels. [6] discusses the beneficial antiviral and anti-inflammatory effects of antimicrobial (cytotoxic) doses of doxycycline (e.g., 100 milligrams twice a day) from the perspectives of four high-risk patients. However, these antiviral and anti-inflammatory benefits may also be available with sub-antimicrobial (prophylactic) dosages of doxycycline (e.g., 20 milligrams twice a day), such as for low-risk patients, or at the onset of COVID infection, or for particular inhibitions (e.g., CD147 expression). Such prophylactic dosages significantly reduce or eliminate any harmful and unintended side effects of cytotoxic dosages. In addition, such dosages still allow many of the pleiotropic effects of doxycycline to counteract the general pathways involved with viral infection, replication, and associated over-exuberant inflammatory response, with associated angiogenic effects. [6]According to [6], doxycycline has no known direct specificity for inhibition of SARS-CoV-2. Doxycycline likely has activity against known coreceptors DPP4/CD26, through demonstrated inhibition of NF-κB and coreceptor CD147/EMMPRIN, necessary for entry of SARS-CoV-2 into T lymphocytes, even at sub-antimicrobial doses. DPP4 appears to have a binding site for NF-κB and inhibition of NF-κB decreases DPP4 expression. DPP4 demonstrates increased expression in older patients and those with diabetes or pulmonary disease, and may account in part for increased morbidity and mortality to COVID-19. Finally, MMP-9 also appears to be required for the virus to traverse the cell wall for infection.

Further, according to [6], once COVID-19 has infected the cell, doxycycline may be able to bind to PLpro, which is responsible for proteolytic cleavage of the replicase polyprotein that releases nonstructural proteins 1, 2, and 3 (Nsp1, Nsp2 and Nsp3), essential for viral replication. It is similarly predicted to bind to 3CLpro or Nsp5, which is cleaved from the polyproteins, causing further cleavage of Nsp4-16, mediating maturation of Nsps essential in the virus life cycle. Doxycycline is an ionophore that binds divalent cations (including zinc) to facilitate cell membrane transport and increase zinc concentration in the cell. Elevated zinc has an inhibitory effect on the replication of SARS-CoV-2. The presumed mechanisms are inhibition of proper processing of replicase proteins and RNA-dependent RNA polymerase (RdRp) activity.

In addition, according to [6], doxycycline may help prevent the cytokine storm following COVID-19 infection presumed to be due to hyperactivity of the immune response to SARS-CoV-2. A primary driver of the anti-inflammatory activity of doxycycline is secondary to its direct inhibition of NF-κB. NF-κB directly regulates IL-6 expression, which is a key driver of the cytokine storm. Doxycycline may also precipitate apoptosis of senescent epithelial cells, which have known increased expression of DPP4/CD26, likely exhibiting increased viral replication in these cells, which leads to induction of the cytokine storm.

Other attempts at using mouthwash for reducing COVID viral load have been largely ineffective. [8] These efforts have been directed primarily at mouthwashes using hydrogen peroxide, povidone iodine (PVP-I, iodopovidone, Betadine®), or chlorhexidine as active ingredients. Their purpose was largely to minimize viral spread (i.e., from patient to professional) in dental office settings. To this end, chlorhexidine and hydrogen peroxide showed little or no inactivation of COVID, while povidone iodine showed some effectiveness (for a specific period) at reducing the viral load in the oral cavity. However, this does not mean povidone iodine is an effective treatment for COVID or practical for preventing COVID infection in more general settings. Rather, povidone iodine appeared to be effective at reducing the number of free virions (from patients) roaming a dental office during and after a relatively brief (no more than two hour) dental procedure. Such oral cavity protection was never established for more than 300 minutes, with two hours or 200 minutes being the usual targeted protection duration for the intended dental office purpose.

By contrast, embodiments of the present disclosure, using a different active ingredient, are targeted at viral infection prevention with a far longer protection (viral load reduction) duration. Embodiments are also directed at stopping progression of the disease. In a study of high-risk individuals treated at the onset of COVID with an antimicrobial dosage of doxycycline mouthwash (400 mg per gargling dosage), none of the group needed hospitalization and all were still alive a month after the time of diagnosis, the treatment effectively preventing progression of the disease. Based on the properties of doxycycline and the targeted delivery of doxycycline mouthwash gargle to the oropharynx entry site, sub-antimicrobial gargle dosages of doxycycline (e.g., 40 mg per gargling dosage) should also be effective at preventing COVID infection.

In some embodiments, a doxycycline-based mouthwash significantly reduces or eliminates the number of COVID virions (viral load) in the mouth. According to [9], doxycycline inhibits COVID viral replication in vitro, such as by inhibiting 3CLpro and RdRp (at the NTP binding channel).

This contrasts with mouthwashes having other active ingredients, such as povidone-iodine, cetylpyridinium chloride, chlorhexidine (e.g., chlorhexidine gluconate), hydrogen peroxide, and benzalkonium chloride. See, e.g., [10] and [13]-[14]. While some of these other mouthwashes have brief effectiveness in the first stage of contamination (e.g., lowering viral load in saliva by killing virions before cell entry), some of the virions remain and are still mostly alive or viable (i.e., capable of causing infection). The effectiveness of these mouthwashes maximizes after about one hour, such as during visits with health care professionals (e.g., dental care teams). While reducing the viral load in saliva can significantly lower the chances of spreading the virus to others during such periods, it does not completely eliminate the risk of infection because some viable virions remain. These other mouthwashes are not very effective at arresting infection after cell entry.

In further detail with reference to [10], chlorhexidine has been shown to reduce salivary viral load in COVID-positive patients for about 60 minutes (i.e., during the first stage of contamination). Further, povidone-iodine and cetylpyridinium chloride are also effective antiseptic mouthwash ingredients for briefly reducing viral load, such as for COVID-positive patients during dental visits. On the other hand, hydrogen peroxide exhibits only minimal reduction in viral load after 30 seconds. In addition, benzalkonium chloride shows mild effectiveness in clinical trials despite good efficacy in laboratory studies. More generally, mouthwashes containing antiseptics such as chlorhexidine, povidone-iodine, cetylpyridinium chloride, hydrogen peroxide, and benzalkonium chloride reduce the number of virions in the first stage of contamination before the virions enter the cell (e.g., in the initial period of infection).

In some embodiments, the proposed mouthwash leverages the principles of (1) penetration inhibition to impede viral fusion and entry into host cells at the host cell membranes after any successful attachment of the virus to, e.g., ACE2, receptors of the host cells, and (2) genetic inhibition to impede subsequent assembly of new virions from replicated genomes of the virus after any successful entry into the host cells. By targeting receptors essential for viral entry and replication, the proposed mouthwash prevents the establishment of infection at the cellular level, which reduces the likelihood of widespread transmission and long-term COVID complications. This preventive approach offers a viable alternative to reactive vaccination strategies, addressing the evolving nature of viral threats more effectively.

In some embodiments, the development of the proposed mouthwash, utilizing doxycycline as a primary active ingredient, focuses on inhibiting viral penetration and replication. Clinical trials targeting high-risk populations, such as individuals over 60 years of age with comorbidities, assess the efficacy of the mouthwash in preventing disease progression and reducing hospitalizations due to respiratory insufficiency. Key endpoints include the incidence of progressive respiratory symptoms and overall survival rates at designated follow-up intervals.

In some embodiments, the proposed mouthwash prevents infection of viruses such as COVID-19. In some embodiments, potential side effects are evaluated and addressed or minimized. For example, antimicrobial overgrowth represents one such concern. In some embodiments, thorough assessments of prior experiences with doxycycline usage in oral formulations are conducted. In some embodiments, rigorous monitoring and surveillance protocols ensure the safety and efficacy of the mouthwash, allowing any adverse events to be addressed promptly in order to optimize patient outcomes.

Considering the evolving landscape of viral threats and the limitations of traditional vaccination approaches, in some embodiments, the development of a targeted genetic inhibitor mouthwash represents a significant stride towards proactive viral prevention. By disrupting viral invasion and replication at the cellular level, embodiments of the present disclosure mitigate the impact of future pandemics and safeguard global public health. Through meticulous research and strategic implementation, embodiments of the proposed mouthwash and COVID prevention techniques offer a compelling solution to the ongoing challenges posed by emerging viral strains.

From the above description of example embodiments of the present disclosure in conjunction with the accompanying drawings, it will be apparent to one of ordinary skill that various modifications can be made without departing from the spirit or scope of the present disclosure. Thus, it is intended that the present disclosure covers such modifications, in particular with regard to the appended claims and their equivalents.

### REFERENCES

[1] Dorobisz et al., Doxycycline in the Coronavirus Disease 2019 Therapy, Therapeutics and Clinical Risk Management 2021:17, pp. 1023-1026.
[2] Dhar et al., Doxycycline for the prevention of progression of COVID-19 to severe disease requiring intensive care unit (ICU) admission: A randomized, controlled, open-label, parallel group trial (DOXPREVENT.ICU), PLOS ONE, https://doi.org/10.1371/journal.pone.0280745, Jan. 23, 2023, 16 pages.
[3] Zhong et al., Editorial: The Mechanisms of Action of Anti-SARS-CoV-2 Drugs, Frontiers in Pharmacology, www.frontiersin.org, April 2022, vol. 13, article 894310, 2 pages.
[4] Toussi et al., Therapeutics for COVID-19, Nature Microbiology, vol. 8, May 2023, pp.771-786, https://doi.org/10.1038/s41564-023-01356-4.
[5] Thacker, Antiviral Activity of Doxycycline, Journal of The Association of Physicians of India (JAPI), vol. 72, issue S2, Feb. 2024, pp. 10-11.
[6] Yates et al., Doxycycline treatment of high-risk COVID-19-positive patients with comorbid pulmonary disease, Therapeutic Advances in Respiratory Disease, vol. 14, 2020, pp. 1-5.
[7] Yates et al., A Proposed Randomized, Double Blind, Placebo Controlled Study Evaluating Doxycycline for the Prevention of COVID-19 Infection and Disease In Healthcare Workers with Ongoing High Risk Exposure to COVID-19, medRxiv 2020.05.11.20098525; doi: https://doi.org/10.1101/2020.05.11.20098525, Protocol No. DOXYPRO-02, Apr. 27, 2020, 59 pages.
[8] Fajardo-Sotamba et al., Efficacy in reducing oral viral load (SARS-CoV-2) of three types of mouthwashes. Literature review, Revista de la Facultad de Odontologia (Universidad Nacional de Cordoba), vol. 32, no. 1, March 2022, pp. 4-10.
[9] Alexpandi et al., Repurposing of Doxycycline to Hinder the Viral Replication of SARS-CoV-2: From in silico to in vitro Validation, Frontiers in Microbiology, vol. 13, article 757418, May 4, 2022, 11 pages.
[10] Balafif et al., Effect of Mouthwash in Reducing the Oral Load of SARS-CoV-2, European Journal of Theoretical and Applied Sciences, vol. 1, no. 3, June 2023, pp. 388-391.
[11] Barbie et al., Doxycycline: new tricks for an old drug, Oncotarget, vol. 6, no. 23, 2015, pp. 19336-19337.
[12] Pambhar et al., Effect of doxycycline and doxycycline with carica papaya on thrombocytopenia and leucopenia in acute dengue fever patients, Journal of Family Medicine and Primary Care, vol. 11, no. 6, June 2022, pp. 3270-3275.
[13] Lin et al., Effectiveness of mouth rinses against COVID-19: a systematic review and network meta-analysis, Journal of Hospital Infection, no. 139, 2023, pp. 175-191.
[14] Alzahrani et al., Mouth rinses efficacy on salivary SARS-CoV-2 viral load: A randomized clinical trial, Journal of Medical Virology, article 28412, 2022, wileyonlinelibrary.com/journal/jmv, https://doi.org/10.1002/jmv.28412, 10 pages.
[15] Alexander et al., Early multidrug treatment of SARS-COV-2 infection (COVID-19) and reduced mortality among nursing home (or outpatient/ambulatory) residents, Medical Hypotheses, www.elsevier.com/locate/mehy, https://doi.org/10.1016/j.mehy.2021.110622, Jun. 5, 2021, 7 pages.
[16] Malek et al., Doxycycline as an Alternative to Azithromycin in Elderly Patients, International Journal of Antimicrobial Agents, vol. 57, 2021, article 106168, https://doi.org/10.1016/j.ijantimicag.2020.106168, 2 pages.

1. (canceled)

2. (canceled)

3. (canceled)

4. (canceled)

5. (canceled)

6. (canceled)

7. (canceled)

8. (canceled)

9. A method of preventing onset or progression of COVID infection in an asymptomatic patient, comprising:
a. gargling a gargling dosage of a preventative gargle at the oropharynx of the asymptomatic patient, the preventative gargle comprising
i. an amount of doxycycline as an active ingredient sufficient for administering the preventative gargle in sub-antimicrobial oral dosages of the doxycycline through gargling at the oropharynx twice a day for several consecutive days, and
ii. sufficient water to dissolve the amount of doxycycline into a liquid solution having sufficient said gargling dosages of the preventative gargle for the several consecutive twice-a-day administrations, each gargling dosage of the preventative gargle having said sub-antimicrobial oral dosage of the doxycycline, and
iii. a container for storing the preventative gargle between the administrations; and
b. repeating the gargling twice a day for the several consecutive days to prevent the onset or progression of COVID infection in the asymptomatic patient.

10. The method of claim 9, wherein the several consecutive days comprises between 5 and 10 consecutive days.

11. The method of claim 10, wherein the several consecutive days comprises 7 consecutive days.

12. The method of claim 9, wherein twice a day comprises every 12 hours.

13. The method of claim 9, wherein
a. the sub-antimicrobial oral dosage comprises between 1 milligram (mg) and 40 mg of the doxycycline, and
b. gargling the gargling dosage of the preventative gargle comprises gargling between 5 milliliters (ml) and 20 ml of the gargle at the oropharynx for at least 30 seconds followed by spitting out the gargled gargle.

14. The method of claim 13, wherein the sub-antimicrobial oral dosage comprises between 1 mg and 4 mg of the doxycycline, and the gargling dosage comprises about 5 ml of the preventative gargle.

15. A method of formulating and using a preventative gargle to prevent onset or progression of COVID infection in an asymptomatic patient, the method comprising:
dissolving doxycycline hyclate in distilled or purified water to create a liquid solution comprising an amount of doxycycline as an active ingredient sufficient for administering the preventative gargle in sub-antimicrobial oral dosages of the doxycycline through gargling at the oropharynx of the asymptomatic patient twice a day for several consecutive days, the liquid solution having sufficient gargling dosages of the preventative gargle for the several consecutive twice-a-day administrations;
dissolving a flavoring agent in the liquid solution to create the preventative gargle;
storing the preventative gargle in a container for use between the administrations;
gargling a gargling dosage of a preventative gargle at the oropharynx of the asymptomatic patient; and
repeating the gargling twice a day for the several consecutive days to prevent the onset or progression of COVID infection in the asymptomatic patient,
wherein each gargling dosage of the preventative gargle comprises said sub-antimicrobial oral dosage of the doxycycline.

16. The method of claim 15, wherein the flavoring agent comprises benzaldehyde.

17. The method of claim 15, further comprising dissolving a preservative in the liquid solution to improve shelf life.

18. The method of claim 15, further comprising dissolving a pH adjuster in the liquid solution to maintain proper pH.

19. The method of claim 15, wherein the sub-antimicrobial oral dosage comprises between 1 milligram (mg) and 40 mg of the doxycycline, and the gargling dosage comprises between 5 milliliters (ml) and 20 ml of the preventative gargle.

20. The method of claim 19, wherein the sub-antimicrobial oral dosage comprises between 1 mg and 4 mg of the doxycycline, and the gargling dosage comprises about 5 ml of the preventative gargle.

21. The method of claim 9, wherein the storage container is amber colored to protect from light and improve shelf life.

22. The method of claim 9, wherein storing the preventative gargle comprises storing the preventative gargle in a cool dark place between the administrations.

23. The method of claim 9, wherein the preventative gargle further comprises a flavoring agent to improve taste.

24. The method of claim 23, wherein the flavoring agent comprises benzaldehyde.

25. The method of claim 9, wherein the preventative gargle further comprises a preservative to improve shelf life.

26. The method of claim 9, wherein the preventative gargle further comprises a pH adjuster to maintain proper pH.
